(19) United States
(12) Patent Application Publication
(10) Pub. No.: US 2009/0112088 A1
(43) Pub. Date: Apr. 30, 2009

(54) ULTRASONIC DIAGNOSTIC APPARATUS, IMAGE DATA GENERATING APPARATUS, ULTRASONIC DIAGNOSTIC METHOD AND IMAGE DATA GENERATING METHOD

(75) Inventors: Hiroyuki Ohuchi, Otawara-Shi (JP); Yasuhiko Abe, Otawara-Shi (JP); Tetsuya Kawagishi, Nasushiobara-Shi (JP)

Correspondence Address: OBLON, SPIVAK, MCCLELLAND MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-shi (JP)

(21) Appl. No.: 12/259,518

(22) Filed: Oct. 28, 2008

(30) Foreign Application Priority Data
Oct. 30, 2007 (JP) 2007-282318

(51) Int. Cl. A61B 8/14; A61B 8/08

(52) U.S. Cl. 600/438; 600/445

(57) ABSTRACT

An ultrasonic diagnostic apparatus includes a displacement measuring unit, a motor information measuring unit, a comparison parameter calculation unit and a parameter image data generating unit. The displacement measuring unit two-dimensionally measures displacements of a myocardial tissue in pieces of ultrasonic image data adjacent in time direction. The motor information measuring unit measures strains and strain rates of the myocardial tissue based on the displacements as motor information. The comparison parameter calculation unit calculates comparison parameters based on the strain rates, or the strains and strain rates. The parameter image data generating unit generates parameter image data using the comparison parameters.

### BACKGROUND OF THE INVENTION

1. Field of the Invention

The present invention relates to an ultrasonic diagnostic apparatus, an image data generating apparatus, an ultrasonic diagnostic method and an image data generating method, and more particularly, to an ultrasonic diagnostic apparatus, an image data generating apparatus, an ultrasonic diagnostic method and an image data generating method which generate parameter image data useful for heart function examination based on time series pieces of ultrasonic image data obtained from an object to which stress echo method is applied.

2. Description of the Related Art

An ultrasonic diagnostic apparatus radiates ultrasonic pulses generated from transducers built in an ultrasonic probe in an object, receives ultrasonic reflected waves generated by a difference of acoustic impedance in an object tissue with the transducers and displays the received reflected waves on a monitor. This diagnostic method is used in a shape diagnostic and a functional diagnostic of biological organs widely since a real-time two-dimensional image is observed easily with a simple operation to contact an ultrasonic probe with surface of a body only (see, for example, Japanese Patent Application (Laid-Open) No. 2005-130877).

The ultrasonic diagnostic method for obtaining biological information by reflected waves from intravital tissues or blood cells makes a rapid advance by two landmark technological developments which are ultrasonic pulse echo method and ultrasonic Doppler method, and a B mode image and a color Doppler image obtained by using the technology mentioned above are necessary for ultrasonic image diagnostic today.

In cardiac functional diagnostic, a method for estimating myocardial motor function using ultrasonic image data acquired under a condition to provide a motor load or a drug load to a patient (hereinafter referred to an object), so-called “stress echo method” is performed widely. In the stress echo method, a method for acquiring B mode image data and color Doppler image data, for example, in time order with changing a load size sequentially based on a stress echo protocol set in advance and displaying pieces of image data obtained under different load states in synchronized with heartbeat is performed generally.

Further, TDI (Tissue Doppler Imaging) method for displaying a moving velocity of a myocardial tissue two-dimensionally by applying the above-mentioned color Doppler method and strain imaging method for two-dimensionally displaying a strain amount measured based on moving velocity information and displacement information of a myocardial tissue obtained by pattern matching to the B mode image data, with overlaying B mode image data and the like, are developed. In addition, a method for estimating myocardial motor function by displaying comparison parameters obtained by comparing two-dimensional strain data acquired from an object before a load to two-dimensional strain data acquired from the object in or after the load as image data (hereinafter referred to parameter image data) is proposed (see, for example, Japanese Patent Application (Laid-Open) No. 2006-26151).

In the conventional cardiac functional diagnostic, estimation of motor function in a cardiac systole (systolic ability) is emphasized specifically, and strain amount (“a strain”) measurement of a myocardial tissue aimed at estimation of a systolic ability is proposed. To the contrary, recently, it proves to be possible to make more early diagnosis of a heart disease by estimation of motor function in a cardiac diastole (extension ability), and it is considered that measurement of “a strain rate” obtained by differentiating “a local strain” of a myocardial tissue in a time direction is efficient for estimation of extension ability.

In this context, according to the above-mentioned method described on Japanese Patent Application (Laid-Open) No. 2006-26151, displaying comparison parameters showing “strain” variations of a myocardial tissue before a load and in or after the load as two-dimensional image data makes it possible to estimate cardiac systolic ability quantitatively and with a high accuracy. On the contrary, there is a problem that it is not necessarily enough to estimate of extension ability aimed at more early diagnosis.

### SUMMARY OF THE INVENTION

The present invention has been made in light of the conventional situations, and it is an object of the present invention to provide an ultrasonic diagnostic apparatus, an image data generating apparatus, an ultrasonic diagnostic method and an image data generating method which can estimate extension ability of a heart quantitatively with a high accuracy based on parameter image data generated by using comparison parameters calculated by comparing “stain rates” in different load states and obtained from a myocardial tissue of an object to which stress echo method is applied.

The present invention provides an ultrasonic diagnostic apparatus which generates parameter image data useful for functional diagnosis of a myocardial tissue based on time series pieces of ultrasonic image data obtained by transmitting and receiving ultrasonic waves to and from an object to which plural mutually different motor or drug loads are sequentially given, the apparatus comprising: a displacement measuring unit configured to two-dimensionally measure displacements of a myocardial tissue in pieces of ultrasonic image data adjacent in time direction; a motor information measuring unit configured to measure strains and strain rates of the myocardial tissue based on the displacements as motor information; a comparison parameter calculation unit configured to calculate comparison parameters based on the strain rates, or the strains and strain rates; and a parameter image data generating unit configured to generate parameter image data using the comparison parameters, in an aspect to achieve the object.

The present invention also provides an image data generating apparatus which generates parameter image data useful for functional diagnosis of a myocardial tissue based on time series pieces of ultrasonic image data obtained by transmitting and receiving ultrasonic waves to and from an object to which plural mutually different motor or drug loads are sequentially given, the apparatus comprising: an ultrasonic image data storing unit configured to store the time series pieces of the ultrasonic image data together with information regarding cardiac time phase and information regarding load state; a displacement measuring unit configured to two-dimensionally measure displacements of a myocardial tissue in pieces of ultrasonic image data adjacent in time direction, read from said ultrasonic image data storing unit; a motor information measuring unit configured to measure strains and strain rates of the myocardial tissue based on the displacements as motor information; a comparison parameter calculation unit configured to calculate comparison parameters based on the strain rates, or the strains and strain rates; and a parameter image data generating unit configured to generate parameter image data using the comparison parameters, in an aspect to achieve the object.

The present invention also provides an ultrasonic diagnostic method for generating parameter image data useful for functional diagnosis of a myocardial tissue based on time series pieces of ultrasonic image data obtained by transmitting and receiving ultrasonic waves to and from an object to which plural mutually different motor or drug loads are sequentially given, the method comprising: two-dimensionally measuring displacements of a myocardial tissue in pieces of ultrasonic image data adjacent in time direction; measuring strains and strain rates of the myocardial tissue based on the displacements as motor information; calculating comparison parameters based on the strain rates, or the strains and strain rates; and generating parameter image data using the comparison parameters, in an aspect to achieve the object.

The present invention also provides an image data generating method for generating parameter image data useful for functional diagnosis of a myocardial tissue based on time series pieces of ultrasonic image data obtained by transmitting and receiving ultrasonic waves to and from an object to which plural mutually different motor or drug loads are sequentially given, the method comprising: storing the time series pieces of the ultrasonic image data together with information regarding cardiac time phase and information regarding load state; two-dimensionally measuring displacements of a myocardial tissue in pieces of ultrasonic image data adjacent in time direction, read from the stored ultrasonic image data; measuring strains and strain rates of the myocardial tissue based on the displacements as motor information; calculating comparison parameters based on the strain rates, or the strains and strain rates; and generating parameter image data using the comparison parameters, in an aspect to achieve the object.

The present invention as described above makes it possible to estimate extension ability of a heart quantitatively with a high accuracy based on parameter image data generated by using comparison parameters calculated by comparing “stain rates” in different load states and obtained from a myocardial tissue of an object to which stress echo method is applied.

### BRIEF DESCRIPTION OF THE DRAWINGS

In the accompanying drawings:

FIG. 1 is a block diagram showing an overall configuration of an ultrasonic diagnostic apparatus according to the first embodiment of the present invention;

FIG. 2 is a diagram showing an example of motor loads and drug loads given to an object to which stress echo method is applied;

FIG. 3 is a block diagram showing a concrete configuration of the transmission/reception system and the ultrasonic image data generating unit included in the ultrasonic diagnostic apparatus according to the first embodiment of the present invention;

FIG. 4 is a diagram explaining tracking processing of a myocardial tissue by cross correlation calculation in the first embodiment;

FIG. 5 is a diagram showing a diastole of a heart set based on an ECG wave in the first embodiment;

FIG. 6 is a flowchart showing a procedure for displaying parameter image data in the first embodiment;

FIG. 7 is a block diagram showing an overall configuration of an ultrasonic diagnostic apparatus according to the second embodiment of the present invention;

FIG. 8 is a block diagram showing a concrete configuration of the comparison parameter calculation unit included in the ultrasonic diagnostic apparatus according to the second embodiment;

FIG. 9 is a diagram showing a concrete example of segmented regions and regions of interest set in a myocardial tissue on a cardiac short axis image in the second embodiment;

FIG. 10 is a diagram showing parameter image data in case where segmented regions are set in a myocardial tissue on a cardiac short axis image in the second embodiment;

FIG. 11 is a flowchart showing a procedure for displaying parameter image data in the second embodiment;

FIG. 12 is a block diagram showing an overall configuration of an image data generating apparatus according to the second embodiment of the present invention; and

FIG. 13 is a flowchart showing a procedure for displaying parameter image data in the third embodiment.

### DESCRIPTION OF THE PREFERRED EMBODIMENTS

Embodiments according to the present invention will now be described below with reference to drawings.

### 1. First Embodiment

In the first embodiment described below, time series pieces of B mode image data are generated by transmitting and receiving ultrasonic waves to and from an object to which plural mutually different motor loads are sequentially given, and subsequently displacements of a myocardial tissue are two-dimensionally measured by tracking processing with pattern matching of the pieces of B mode image data. Then, “strains” and “strain rates” of the myocardial tissue are measured based on a spatial gradient and a time variation of the displacements. Furthermore, the maximum strains and the maximum strain rates are extracted from time series strains and strain rates measured based on B mode image data in a diastole. Then, comparison parameters are calculated using the two-dimensional maximum strains and maximum strain rates extracted with regard to the mutually different two motor loads and comparison image data is generated based on the obtained comparison parameters.

Note that, in the following embodiment, acquiring B mode image data as ultrasonic image data with regard to an object provided a motor load and generating desired parameter image data based on the B mode image data will be described. The object may be also provided a drug load instead of a motor load and acquiring tissue Doppler image data and/or color Doppler image data under the color Doppler method as ultrasonic image data is also possible.

### (Configuration of Apparatus)

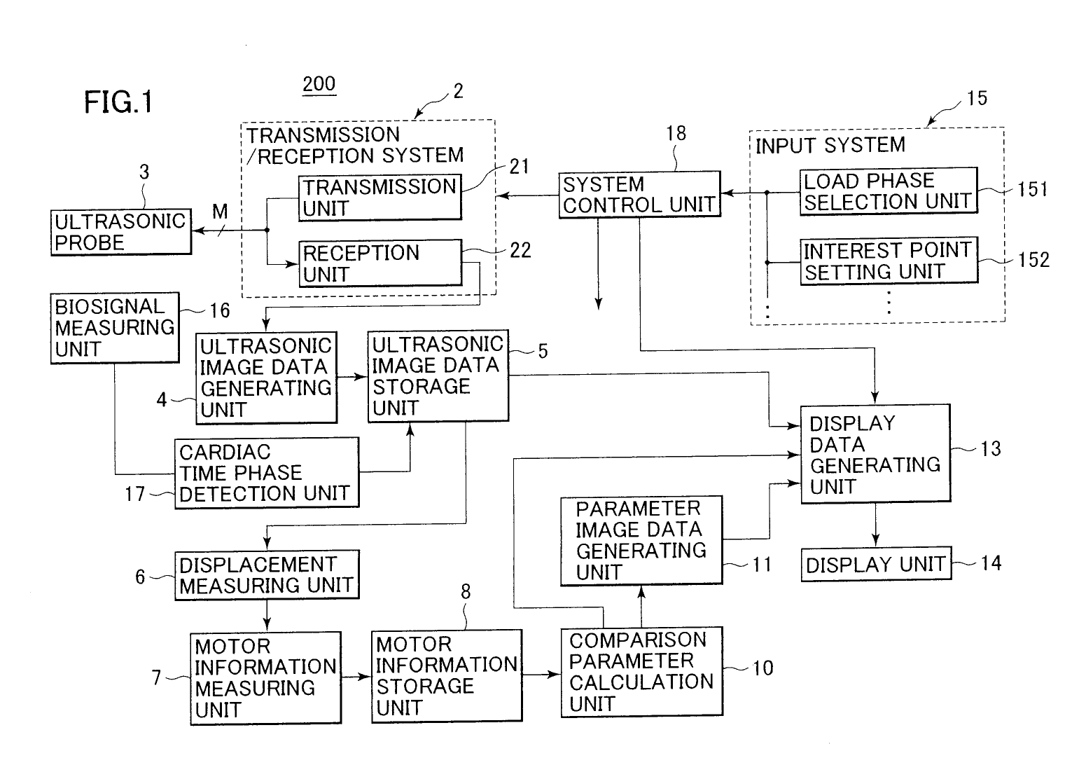

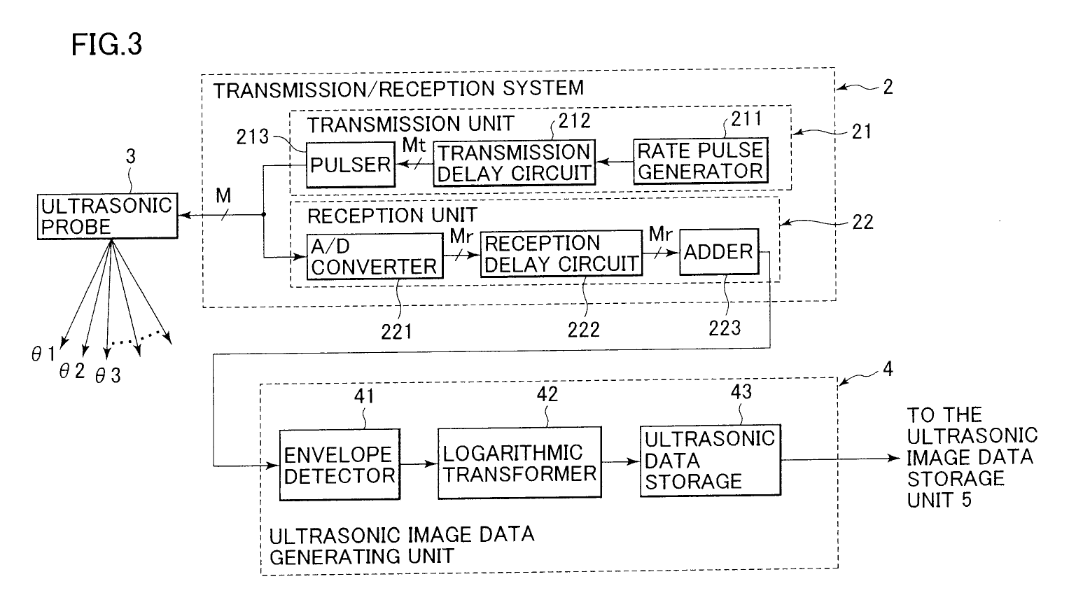

A configuration of an ultrasonic diagnostic apparatus according to the first embodiment of the present invention and fundamental operation of each unit of the apparatus will be described with reference to FIGS. 1 to 5. FIG. 1 is a block diagram showing an overall configuration of the ultrasonic diagnostic apparatus according to the present embodiment. FIG. 3 is a block diagram showing a concrete configuration of a transmission/reception system and an ultrasonic image data generating unit included in the ultrasonic diagnostic apparatus.

An ultrasonic diagnostic apparatus 200 according to the present embodiment shown in FIG. 1 includes an ultrasonic probe 3, a transmission/reception system 2, an ultrasonic image data generating unit 4 and an ultrasonic image data storage unit 5. The ultrasonic probe 3 has plural transducers each configured to transmit an ultrasonic pulse (a transmission ultrasonic wave) to an object of which a load state by motor load (hereinafter referred to a load phase) is updated sequentially, and convert an ultrasonic reflected wave (a reception ultrasonic wave) acquired from the object into an electric signal (a reception signal). The transmission/reception system 2 is configured to supply drive signals, to the transducers of the ultrasonic probe 3, for transmitting ultrasonic pulses toward a predetermined direction in the object, and to phase and add reception signals respectively corresponding to plural channels, obtained from the transducers. The ultrasonic image data generating unit 4 is configured to perform signal processing of the phased and added reception signals to generate B mode image data. The ultrasonic image data storage unit 5 is configured to add information regarding load phase and cardiac time phase information of a diastole or a systole supplied from the after-mentioned cardiac time phase detection unit 17 to the time-series B mode image data outputted from the ultrasonic image data generating unit 4 and to store the time-series B mode image data.

The ultrasonic diagnostic apparatus 200 also includes a displacement measuring unit 6, a motor information measuring unit 7, a motor information storage unit 8 and a comparison parameter calculation unit 10. The displacement measuring unit 6 is configured to measure local displacements of myocardial tissue on the respective time-series pieces of B mode data supplied from the ultrasonic image data storage unit 5. The motor information measuring unit 7 is configured to measure “strains” and “strain rates” of the myocardial tissue as motor information based on spatial gradients and temporal variations of the above-mentioned displacements. The motor information storage unit 8 is configured to add the load phase information and the cardiac time phase information to the above-mentioned motor information acquired two-dimensionally with corresponding to the respective time-series pieces of the B mode image data and to store the motor information. The comparison parameter calculation unit 10 is configured to extract “the maximum strain” and “the maximum strain rate” each representing the maximum value or the minimum value from time-series “strains” and “strain rates” at a predetermined portion during a diastole in each of the first load phase (load phase ni) and the second load phase (load phase nj) read from the motor information storage unit 8, and to calculate a comparison parameter based on “the maximum strain” and “the maximum strain rate”.

The ultrasonic diagnostic apparatus 200 also includes a parameter image data generating unit 11, a display data generating unit 13, a display unit 14, an input system 15, a bio-signal measuring unit 16, the cardiac time phase detection unit 17 and a system control unit 18. The parameter image data generating unit 11 is configured to generate parameter image data based on a comparison parameter calculated by the above-mentioned comparison parameter calculation unit 10. The display data generating unit 13 is configured to generate display data by overlaying the B mode image data with the parameter image data. The display unit 14 is configured to display the obtained display data. The input system 15 is configured to perform inputting of object information, setting of respective conditions for generating ultrasonic image data, parameter image data and display data, selection of a load phase ni and a load phase nj, selection of estimation function of extension ability, inputting of various command signals and the like. The bio-signal measuring unit 16 is configured to measure an ECG wave form of an object. The cardiac time phase detection unit 17 is configured to detect a cardiac time phase during a diastole or a systole using an R wave on an ECG wave form as a reference. The system control unit 18 is configured to overall-control the above-mentioned respective units.

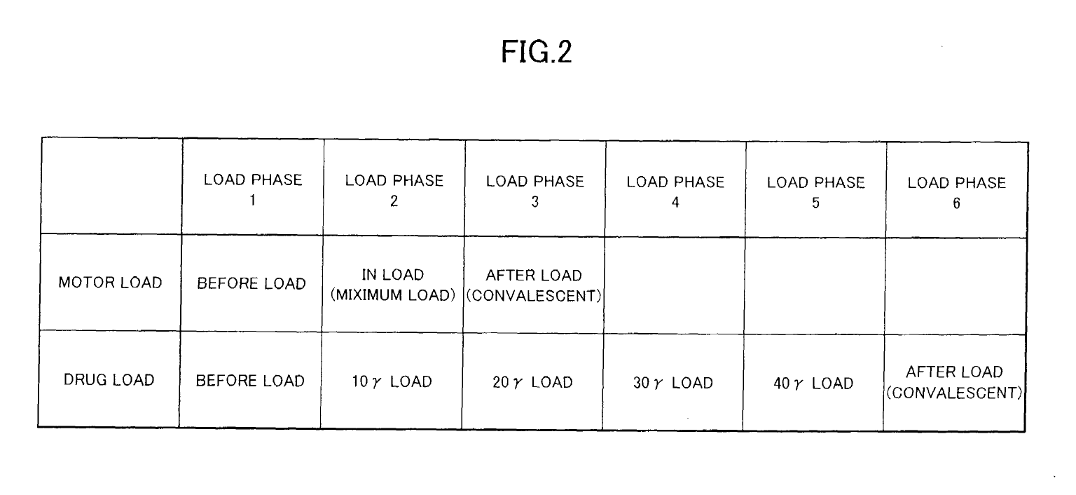

Here, a concrete example of motor loads and drug loads given to a normal object under the stress echo method is shown in FIG. 2. As phases of motor load, for example, a load phase 1 before a load, a load phase 2 in the maximum load, and a load phase 3 after a load which is convalescent are set in advance as a protocol for the stress echo method. As phases of drug load, a load phase 1 before a load, a load phase 2 to a load phase 5 administered 10 γ to 40 γ of drug (for example dobutamine) sequentially, and a load phase 6 after a load which is convalescent are set.

Then, in the stress echo method by motor load, a method for generating parameter image data based on a comparison result between motor information measured in the load phase 1 before load and motor information measured in the load phase 2 is performed generally. In the present embodiment hereinafter described, the case that the load phase 1 by motor load is the load phase ni and the load phase 2 is the load phase nj will be described, however, is not limited to.

Then, the above-mentioned respective units included in the ultrasonic diagnostic apparatus 200 according to the present embodiment will be described in further detail.

The ultrasonic probe 3 in FIG. 1 is configured to have two-dimensionally arrayed M transducers not shown in the figure at the tip part and transmit and receive ultrasonic waves with contacting the tip part with surface of an object body. Each transducer is an electroacoustic conversion element and has a function to convert an electric pulse (a drive signal) into an ultrasonic pulse (a transmission ultrasonic wave) at the time of transmission and to convert an ultrasonic reflected wave (a reception ultrasonic wave) into an electric reception signal at the time of reception. Then, the respective transducers are connected with the transmission/reception system 2 through M channels of multicore cables not shown in the figure. Note that, in the present embodiment, a case of using the ultrasonic probe 3 of which M transducers are arranged one-dimensionally for a sector scan will be described. An ultrasonic probe corresponding to a scan such as a linear scan and a convex scan may be used.

The transmission/reception system 2 shown in FIG. 3 includes a transmission unit 21 and a reception unit 22. The transmission unit 21 is configured to supply drive signals to the transducers of the ultrasonic probe 3. The reception unit 22 is configured to phase and add the reception signals obtained from the transducers.

The transmission unit 21 includes a rate pulse generator 211, a transmission delay circuit 212 and a pulser 213. The rate pulse generator 211 is configured to generate a rate pulse to determine a repetition period of a transmission ultrasonic wave and to supply the rate pulse to the transmission delay circuit 212. The transmission delay circuit 212 includes Mt transducers used for transmission and the same number of the independent delay circuits and is configured to provide a delay time for focus for focusing a transmission ultrasonic wave on a predetermined depth and a delay time for deflection for transmission toward a predetermined direction θp to each rate pulse and to supply the rate pulse to the pulser 213. The pulser 213 has the same number of independent pulsers as that of the transmission delay circuit 212 and is configured to drive Mt (Mt≦M) transducers, selected from the M transducers arranged in the ultrasonic probe 3 as for transmission, with the drive signals generated based on the rate pulse and to radiate a transmission ultrasonic waves in the body of the object.

Meanwhile, the reception unit 22 includes Mr channels of A/D converters 221 and reception delay circuits 222, corresponding to Mr (Mr≦M) transducers selected from the M transducers integrated into the ultrasonic probe 3 as for reception, and an adder 223. The Mr channels of reception signals supplied from the transducers for reception are converted into digital signals in the A/D converters 221 and transmitted to the reception delay circuits 222.

The reception delay circuits 222 are configured to provide delay times for focus for focusing a reception ultrasonic wave from a predetermined depth and delay times for deflection for setting reception directionality to a predetermined direction θp to the respective Mr channels of reception signals outputted from the A/D converters 221. The adder 223 is configured to add the reception signals from the reception delay circuits 222. That is, the reception signals obtained from the predetermined direction are phased and added by the reception delay circuits 222 and the adder 223.

The ultrasonic image data generating unit 4 has a function to perform signal processing of the phased and added reception signal outputted from the adder 223 in the reception unit 22 to generate B mode image data. The ultrasonic image data generating unit 4 includes a envelop detector 41, a logarithmic transformer 42 and an ultrasonic data storage 43. The envelope detector 41 is configured to perform envelop detection of the reception signal. The logarithmic transformer 42 is configured to perform logarithmic transformation of the reception signal after the envelop detection to generate B mode data. The ultrasonic data storage 43 is configured to generate two-dimensional B mode image data as ultrasonic image data by storing the obtained B mode data with corresponding to a direction of ultrasonic wave transmission/reception. Note that, the order of the envelop detector 41 and the logarithmic transformer 42 can be replaced. Then, time-series pieces of B mode image data, corresponding to respective load phases, generated in the ultrasonic image data generating unit 4 are stored in the ultrasonic image data storage unit 5 shown in FIG. 1 with adding information regarding load phase supplied from the system control unit 18 and cardiac time phase information of a diastole or a systole supplied from the cardiac time phase detection unit 17 as incidental information.

Meanwhile, the displacement measuring unit 6 shown in FIG. 1 is configured to perform tracking processing by pattern matching between two pieces of the B mode image data mutually adjacent in the time direction (that is, the B mode image data A1 acquired at a reference time t0 and the B mode image data A2 acquired at a time t0+δT) from the time-series pieces of the B mode image data stored in the ultrasonic image data storage unit 5 to measure a movement distance (displacement) at the time δT of a myocardial tissue shown in the B mode image data.

For example, plural interest points are set at predetermined intervals in a myocardial tissue area of the B mode image data A1 preceding in time, in addition, two-dimensional correlation regions of which centers are the respective interest points are set. Then, cross-correlation calculation between corresponding pixels is performed with relatively moving image information (template) on each correlation region to the B mode image data A2 following the B mode image data A1. Further, a local displacement of the myocardial tissue shown by an interest point is measured by detecting a moving direction and a movement distance of the interest point so that a correlation value becomes max. Similar measurements are performed with regard to other interest points set in the B mode image data A1, in addition, performed with regard to plural respective pieces of B mode image data following the B mode image data A2.

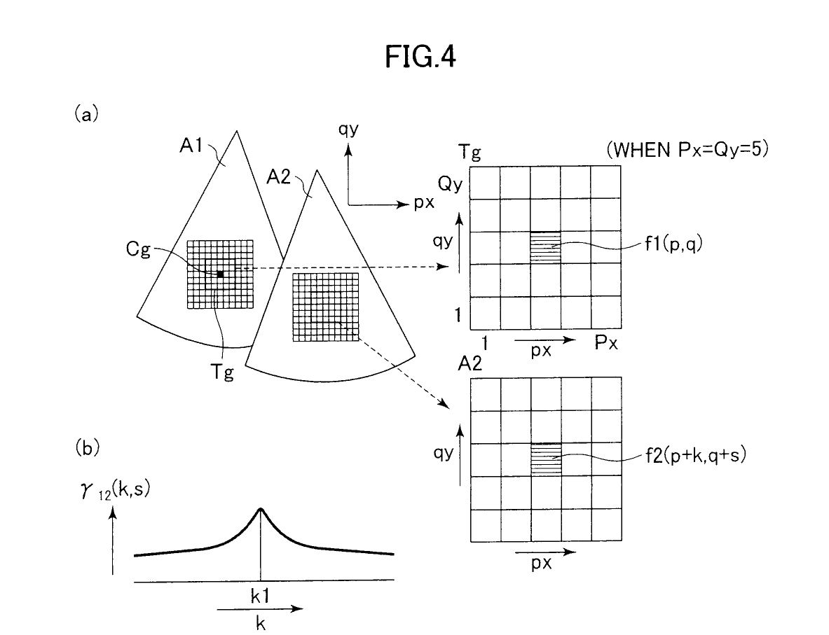

The tracking processing of a myocardial tissue by cross-correlation calculation will be explained more precisely using FIG. 4. The interest point Cg shown in FIG. 4(a) is one of plural interest points set at predetermined intervals in a myocardial tissue area of the B mode image data A1. When a pixel value of a template Tg, having the predetermined number No. (No.=PxQy) of pixels, with, of which the interest point Cg is center is denoted as f1(px, qy), and a pixel value of the B mode image data A2 is denoted as f2(px, qy), a displacement of the myocardial tissue, after the time δT, corresponding to a interest point Cg in the B mode image data A1 can be measured by calculating the cross-correlation coefficient γ12 (k, s) with the following expression (1).

$${γ}_{12} ( k , s ) = \frac{1}{No {σ}_{1} {σ}_{2}} {\sum }_{Px = 1}^{Px} {\sum }_{qy = 1}^{Qy} ( f 1 ( px , qy ) - \overline{f 1} ) ( f 2 ( px + k , qy + s ) - \overline{f 2} ) \\ \overline{f 1} = \frac{1}{No} {\sum }_{Px = 1}^{Px} {\sum }_{qy = 1}^{Qy} f 1 ( px , qy ) \\ \overline{f 2} = \frac{1}{No} {\sum }_{Px = 1}^{Px} {\sum }_{qy = 1}^{Qy} f 2 ( px + k , qy + s ) \\ {σ}_{1}^{2} = \frac{1}{No} {\sum }_{Px = 1}^{Px} {\sum }_{qy = 1}^{Qy} {( f 1 ( px , qy ) - \overline{f 1} )}^{2} \\ {σ}_{2}^{2} = \frac{1}{No} {\sum }_{Px = 1}^{Px} {\sum }_{qy = 1}^{Qy} {( f 2 ( px , qy ) - \overline{f 2} )}^{2} \\ No = PxQy \qquad ( 1 )$$

Note that, the above-mentioned Px and Qy denote the numbers of pixels of the template Tg in the px direction and the qy direction respectively, and the interest point Cg set in the B mode image data A1 normally locates in the nearly center of the template Tg. In case that γ12 (k, s) has a maximum value when k=k1 (FIG. 4 (b) reference) and s=s1 (not shown) by the cross-correlation calculation, it is represented that the local myocardial tissue shown as the interest point Cg of the B mode image data A1 is displaced by k1 pixels in the px direction and by s1 pixels in the qy direction respectively in the B mode image data A2.

The tracking processing mentioned above is performed with regard to all interest points set in the myocardial tissue area of the B mode image data A1 and the displacements in the B mode image data A2 of the local myocardial tissue areas indicated by the respective interest points are measured. In addition, the similar tracking processing of respective plural pieces of the B mode image data following the B mode image data A2 is performed and the displacements of the myocardial tissue in the pieces of the B mode image data are measured.

Back to FIG. 1, the motor information measuring unit 7 is configured to measure local “strains” based on spatial gradients of the displacement amounts at the plural interest points measured in the displacement measuring unit 6, moreover, to measure “strain rates” based on temporal variations (that is, differential values in the time direction) of the “strains” measured with regard to the respective time-series pieces of B mode image data. Then, motor information including the time-series “strains” and “strain rates” measured at the respective plural interest points set in the myocardial tissue area is stored in the motor information storage unit 8 with the cardiac time phase information and the load phase information added to the B mode image data as incidental information. That is, the motor information of two-dimensional “strains” and “strain rates” measured in time-series in plural load phases is stored together with the cardiac time phase information and the load phase information in the motor information storage unit 8.

Meanwhile, the comparison parameter calculation unit 10 includes a maximum value extracting part and a calculation part to be not shown in the figure. The maximum value extracting part is configured to extract the time-series “strain rates” SR(i, t, x, y) and SR(j, t, x, y) appending cardiac time phase information corresponding to arbitrary periods during a diastole and information indicating either load phase ni or load phase nj as incidental information and the time-series “strains” S(j, t, x, y) appending cardiac time phase information corresponding to arbitrary periods during a diastole and information indicating the load phase nj as incidental information from the motor information stored in the motor information storage unit 8, based on information for selecting the load phase ni and the load phase nj (that is, the load phase 1 and the load phase 2 by a motor load) supplied from the input system 15 and information for applying estimation function of extension ability.

Then, the maximum value extracting part is configured to extract “the maximum strain rates” SRmax(i, x, y) and “the maximum strain rates” SRmax(j, x, y) and “the maximum strains” Smax(j, x, y) each representing the maximum value or the minimum value in the time direction, from the respective time-series “strain rates” SR(i, t, x, y) and “strain rates” SR(j, t, x, y) and “strains” S(j, t, x, y) measured at the interest points Cg(x, y).

Meanwhile, the calculation part is configured to calculate comparison parameters K1(x, y) to K3(x, y) by substituting the above-mentioned “maximum strain rates” SRmax(i, x, y) and “maximum strain rates” SRmax(j, x, y) and “maximum strains” Smax(j, x, y) into the following expression (2).

$$K 1 ( x , y ) = \frac{S max ( j , x , y ) \cdot SR max ( j , x , y )}{SR max ( i , x , y )} \\ K 2 ( x , y ) = S max ( j , x , y ) \cdot \{ SR max ( j , x , y ) - SR max ( i , x , y ) \} \\ K 3 ( x , y ) = \frac{S max ( j , x , y ) \cdot \{ SR max ( j , x , y ) - SR max ( i , x , y ) \}}{SR max ( i , x , y )} \qquad ( 2 )$$

The parameter image data generating unit 11 is configured to generate parameter image data using either of the above-mentioned comparison parameters K1(x, y) to K3(x, y) calculated two-dimensionally by the comparison parameter calculation unit 10.

The display data generating unit 13 is configured to perform color transformation of pixel values (comparison parameters) of parameter image data, supplied from the parameter image data generating unit 11, based on a pixel value-color transformation format set preliminarily, in addition, to generate display data by overlaying comparison parameter image data after the color transformation with B mode image data supplied from the ultrasonic image data storage unit 5. In this case, the display data generating unit 13 is configured to compare each of the two-dimensional “maximum strains” Smax(j, x, y) supplied from the comparison parameter calculation unit 10 with a predetermined threshold supplied from the input system 15 and to perform color transformation of a pixel value, of the parameter image data, corresponding to “the maximum strain” Smax(j, x, y) more than the threshold, based on a predetermined pixel value-color transformation format. In addition, the display data generating unit 13 is configured to transform a pixel value, of the parameter image data, corresponding to “the maximum strain” Smax(j, x, y) not more than the threshold, based on another different pixel value-color transformation format.

The display unit 14 includes a data transformation part and a monitor to be not shown in the figure. The data transformation part is configured to perform D/A transformation and display format transformation of the above-mentioned display data supplied from the display data generating unit 13 and to display the transformed data on the monitor. For example, each pixel showing “the maximum strain” more than the predetermined threshold and of which sign is plus (+) is displayed in warm colors such as red while each pixel of which sign is minus (−) is displayed in cold colors such as blue, by the display data generating unit 13 and the display unit 14. Each absolute value of the pixel values is identified by luminance/brightness/color phase and the like. Meanwhile, for example, each pixel showing “the maximum strain” not more than the threshold and of which sign is minus (−) is displayed in purple. By applying the display method mentioned above, a healthy portion in a myocardial tissue is displayed in warm colors, an extension ability depression portion due to a mild or moderate ischemia and the like is displayed in cold colors and a contractional ability depression portion due to a severe ischemia, total necrosis and the like is displayed in purple.

The input system 15 is an interactive interface which includes input devices such as a display panel, a keyboard, various switches, selection buttons, a mouse and a trackball. The input system 15 includes a load phase selection unit 151 and an interest point setting unit 152. The load phase selection unit 151 is configured to select desired load phase ni and load phase nj from plural load phases. The interest point setting unit 152 is configured to set interest points at predetermined intervals in a myocardial tissue. Inputting of object information, setting of respective conditions for generating ultrasonic image data, parameter image data and display data, selection of estimation function of extension ability, setting of respective thresholds to “the maximum strain” and an ECG wave form, inputting of various command signals and the like are also performed using the above-mentioned input devices and/or display panel included in the input system 15.

The biosignal measuring unit 16 has a function to measure an ECG waveform of an object. The biosignal measuring unit 16 includes an electrode for measurement which is placed on body surface of an object and detects an ECG waveform, an amplifier circuit configured to amplify the ECG waveform detected by the electrode for measurement to a predetermined amplitude and an A/D converter configured to convert the amplified ECG waveform into a digital signal (all of them are not shown in the figure).

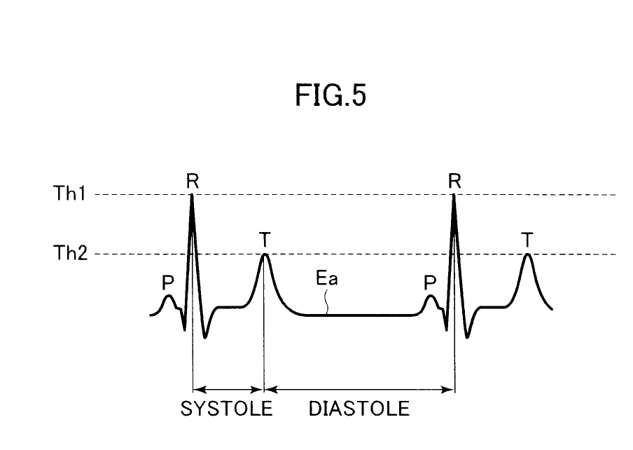

Then, as shown in FIG. 5 with scheme, the cardiac time phase detection unit 17 is configured to set a predetermined thresholds Th1 and Th2 to an ECG waveform Ec after A/D conversion supplied from the biosignal measuring unit 16 to detect an R wave and a T wave, in addition, to detect cardiac time phases during a systole from an R wave to a T wave and a diastole from a T wave to an R wave. Then, the cardiac time phase information is added to the time-series pieces of B mode image data generated in the ultrasonic image data generating unit 4, together with the load phase information supplied from the system control unit 18, and stored in the ultrasonic image data storage unit 5.

The system control unit 18 includes a CPU and a storage circuit to be not shown in the figure. Information regarding various settings/selecting conditions from the input system 15 is stored in the above-mentioned storage circuit. Then, the CPU is configured to overall-control respective units in the ultrasonic diagnostic apparatus 200 based on the above-mentioned information stored in the storage circuit, to generate the B mode image data and the parameter image data and to generate and display the display data based on the B mode image data and the parameter image data.

### (Procedure for Displaying Parameter Image Data)

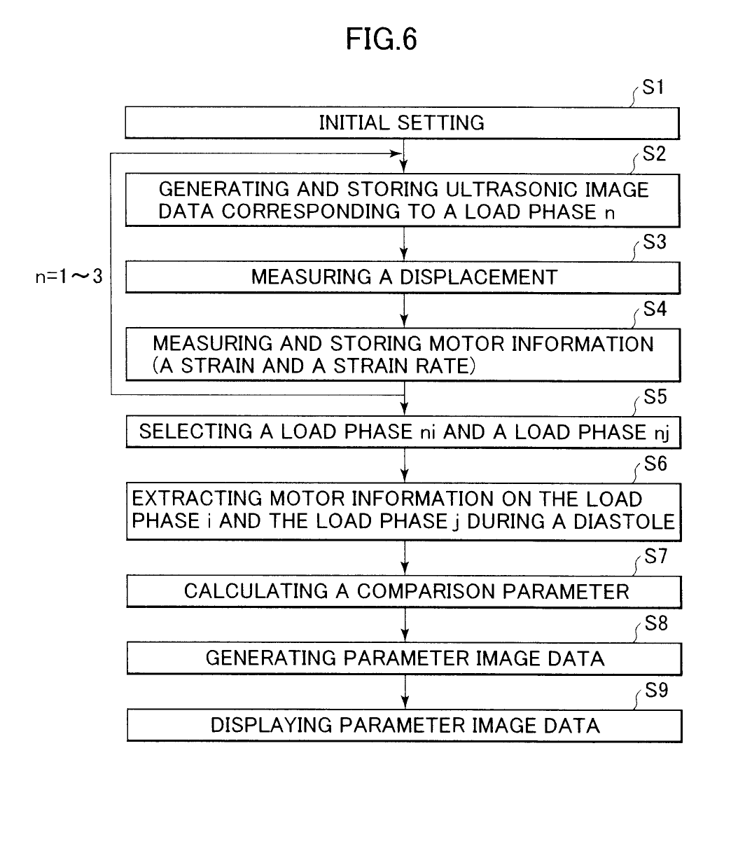

Then, a procedure for displaying parameter image data in the present embodiment will be described with reference to the flowchart on FIG. 6.

Prior to generating parameter image data, an operator of the ultrasonic diagnostic apparatus 200 places the electrode for measurement included in the biosignal measuring unit 16 on a predetermined portion of an object in a static state (the load phase 1 by motor load) after inputting of object information, selection of B mode image data as ultrasonic image data, setting of respective conditions for generating ultrasonic image data, parameter image data and display data, selection of estimations function of extension ability, setting of thresholds to “the maximum strain” and an ECG waveform and the like with the input system 15 (step S1 in FIG. 6).

When the above-mentioned initial setting is completed, the operator inputs a measuring start command of motor information from the input system 15 with fixing the tip (ultrasonic wave transmission/reception surface) of the ultrasonic probe 3 on body surface of the object in the load phase 1 (before load). Then, motor information measurement of a myocardial tissue with using B mode image data starts by supplying the command signal to the system control unit 18.

In acquiring the B mode image data corresponding to the load phase 1, the rate pulse generator 211 in the transmission unit 21 shown in FIG. 3 generates rate pulses by dividing a reference signal supplied from the system control unit 18 and to supply the rate pulses to the transmission delay circuits 212. The transmission delay circuits 212 provide delay times for focus for focusing an ultrasonic wave to a predetermined depth and delay times for deflecting for transmitting the ultrasonic wave in the initial transmission/reception direction θ1 to the rate pulses and supply the rate pulses to the Mt channels of the pulsers 213. Subsequently, the pulsers 213 generate drive signals based on the rate pulses supplied from the transmission delay circuits 212 and supply the drive signals to the Mt transducers for transmission of the ultrasonic probe 3 to radiate a transmission ultrasonic wave in the object.

A part of the radiated transmission ultrasonic wave is reflected at an organ interface or a tissue of the object having a different acoustic impedance and received by the Mr transducers for reception set in the ultrasonic probe 3 to be converted into the Mr channels of electronic reception signals. Subsequently, the reception signals are converted into digital signals in the A/D converter 221 included in the reception unit 22. Additionally, delay times for focus for focusing the reception ultrasonic wave from the predetermined depth and delay times for deflection for setting large reception directionality to the reception ultrasonic wave from the transmission/reception direction θ1 are given to the digital reception signals in the Mr channels of the reception delay circuits 222. Subsequently, the digital reception signals are phased and mutually added in the adder 223.

Then, the envelope detector 41 and the logarithmic transformer 42 in the ultrasonic image data generating unit 4, to which the phased and added reception signal is supplied, perform envelop detection and logarithmic transformation of the reception signal to generate B mode data respectively. The obtained B mode data is stored in the ultrasonic data storage 43 with corresponding to the transmission/reception direction.

When generation and storage of the B mode data corresponding to the transmission/reception direction θ1 are completed, the system control unit 18 controls the delay times for the transmission delay circuit 212 included in the transmission unit 21 and for the reception delay circuit 222 included in the reception unit 22 so as to perform a two-dimensional scan by transmitting and receiving an ultrasonic wave to and from each of respective transmission/reception directions θp (θp=θ1+(p−1)Δθ(p=2˜P) obtained by sequentially updating the transmission/reception direction in the θ direction by Δθ, with the similar procedure. Then, the pieces of B mode data obtained with regard to the transmission/reception directions are also stored in the ultrasonic data storage 43 with corresponding to the transmission/reception directions respectively. That is, the initial pieces of the B mode image data is generated in the ultrasonic data storage 43, in addition, the time-series pieces of the B mode image data generated by repeating the above-mentioned two-dimensional scan are supplied to the ultrasonic image data storage unit 5.

On the other hands, the cardiac time phase detection unit 17 sets predetermined thresholds on the ECG waveform, after A/D conversion, supplied from the biosignal measuring unit 16 to detect an R wave and a T wave, moreover, and detect a cardiac time phase during a systole from an R wave to a T wave and a cardiac time phase during a diastole from a T wave to an R wave. Then, the pieces of cardiac time phase information are supplied to the ultrasonic image data storage unit 5.

The ultrasonic image data storage unit 5 adds the cardiac time phase information supplied from the cardiac time phase detection unit 17 and information regarding the load phase 1 supplied from the system control unit 18 to the time-series pieces of B mode image data supplied from the ultrasonic image data generating unit 4 and stores the time-series pieces of B mode image data (step S2 in FIG. 6).

On the other hands, the displacement measuring unit 6 sequentially extracts two pieces of B mode image data adjacent in the time direction from the time-series pieces of B mode image data stored in the ultrasonic image data storage unit 5. In this time, the operator who observes the initial piece of B mode image data displayed on the display unit 14 through the display data generating unit 13 sets plural interest points Cg in a myocardial tissue area of the B mode image data using the interest point setting unit 152 in the input system 15. The displacement measuring unit 6 which received the setting information performs tracking processing by pattern matching with centering on respective interest points Cg to measure movement distances (displacements) of the myocardial tissue shown in the B mode image data (step S3 in FIG. 6).

Subsequently, the motor information measuring unit 7 measures local “strains” based on spatial gradients of the two-dimensional displacements measured in the displacement measuring unit 6 and measures “strain rates” based on temporal variations of the “strains” measured with regard to the respective time-series pieces of the B mode image data. Then, the motor information measuring unit 7 adds the cardiac time phase information and the load phase information (the information regarding load phase 1), which are incidental information of the B mode image data, to the motor information including the time-series “strains” and “strain rates” measured in the myocardial tissue area, and stores the information in the motor information storage unit 8 (step S4 in FIG. 6).

When generating and storing the piece of B mode image data corresponding to the load phase 1 and measuring and storing the motor information are completed, generating and storing pieces of B mode image data and measuring and storing pieces of motor information are performed to the object in the load phase 2 and the load phase 3 with the similar procedure (from step S2 to step S4 in FIG. 6).

When measuring and storing the pieces of motor information corresponding to the load phases 1 to 3 by motor load are completed, the operator selects a load phase ni and a load phase nj (for example, the load phase 1 and the load phase 2) necessary for generating parameter image data by the load phase selection unit 151 included in the input system 15 (step S5 in FIG. 6). The comparison parameter calculation unit 10 extracts time-series “strain rates” SR(i, t, x, y) and/or “strain rates” SR(j, t, x, y) attaching cardiac time phase information during an arbitrary period in a diastole and information indicating either load phase ni or load phase nj as incidental information and time-series “strains” S(j, t, x, y) attaching the cardiac time phase information during the arbitrary period and information indicating the load phase nj as incidental information from the pieces of motor information stored in the motor information storage unit 8, based on selecting information of the load phases and information selecting the estimation function of extension ability supplied through the system control unit 18 (step S6 in FIG. 6).

Subsequently, the comparison parameter calculation unit 10 extracts “the maximum strain rates” SRmax(i, x, y), “the maximum strain rates” SRmax(j, x, y) and “the maximum strains” Smax(j, x, y), each representing the maximum value or the minimum value in the time direction, from the respective time-series “strain rates” SR(i, t, x, y), “strain rates” SR(j, t, x, y) and “strains” S(j, t, x, y) measured at the plural interest points Cg (x, y) in the B mode image data. Then, the comparison parameter calculation unit 10 calculates comparison parameters K1 (x, y) to K3(x, y) based on “the maximum strain rates” and “the maximum strains” (step S7 in FIG. 6). Then, the parameter image data generating unit 11 generates parameter image data using at least one of the above-mentioned comparison parameters K1(x, y) to K3 (x, y) calculated two-dimensionally by the comparison parameter calculation unit 10 (step S8 in FIG. 6).

Meanwhile, the display data generating unit 13 compares each of the two-dimensional “maximum strains” Smax(j, x, y) supplied from the comparison parameter calculation unit 10 to the predetermined threshold supplied from the input system 15 and performs color transformation of each pixel value (comparison parameter) of the parameter image data corresponding to “the maximum strain” Smax (j, x, y) more than the threshold, based on a predetermined pixel value-color transformation format. Additionally, the display data generating unit 13 transforms each pixel value of the parameter image data corresponding to “the maximum strain” Smax (j, x, y) not more than the threshold, based on another different transformation format. Then, the display data generating unit 13 generates display data by overlaying the B mode image data supplied from the ultrasonic image data storage unit 5 on the comparison parameter image data after color transformation and displays the display data on the monitor of the display unit 14 (step S9 in FIG. 6).

### (Modification)

Note that, a case where the comparison parameter calculation unit 10 calculates the comparison parameters K1 to K3 using “the maximum strain rates” SRmax(I, x, y) in the load phase ni, and “the maximum strain rates” SRmax(j, x, y) and “the maximum strains” Smax(j, x, y) in the load phase nj was described in the above-mentioned embodiment. However, the comparison parameters K1 to K3 may be calculated by substituting “the maximum strain rates” SRmax(I, x, y) in the load phase ni and “the maximum strain rates” SRmax(j, x, y) in the load phase nj in the following expression (3).

$$K 1 ( x , y ) = \frac{SR max ( j , x , y )}{SR max ( i , x , y )} \\ K 2 ( x , y ) = SR max ( j , x , y ) - SR max ( i , x , y ) \\ K 3 ( x , y ) = \frac{SR max ( j , x , y ) - SR max ( i , x , y )}{SR max ( i , x , y )} \qquad ( 3 )$$

According to the first embodiment mentioned above, a quantitative estimation with regard to extension ability of a myocardial tissue can be performed with high accuracy by generating parameter image data based on myocardial local strain rate information, or strain rate information and strain information obtained in the load phase ni and the load phase nj of the object to which a stress echo method is applied.

Especially, since a comparison parameter is calculated using “the maximum strain rate” and “the maximum strain” extracted from time-series “strain rates” and “strains” and desired parameter image data is generated based on the comparison parameter in the above-mentioned embodiment, stable parameter image data can be obtained.

Further, a normal myocardial tissue and a myocardial tissue with a decreased extension ability can be also observed specifically and easily by performing color display of parameter image data based on a pixel value-color transformation format with a cold color and a warm color corresponding to signs of the comparison parameters.

Additionally, it is possible to distinctly observe a normal myocardial tissue, a myocardial tissue with a decreased extension ability due to a mild or moderate ischemia and the like and a myocardial tissue of which contractile ability is decreased drastically due to a severe ischemia, total necrosis and the like by performing color display of each pixel value of parameter image data corresponding to “the maximum strain” more than a predetermined threshold, based on the above-mentioned pixel value-color transformation format and of each pixel value of the parameter image data corresponding to “the maximum strain” not more than the threshold, based on another transformation format with comparing the above-mentioned “maximum strain” with the threshold.

### 2. Second Embodiment

Subsequently, the second embodiment of the present invention will be described below. In the second embodiment, time series pieces of B mode image data are generated by transmitting and receiving ultrasonic waves to and from an object to which plural mutually different motor loads are sequentially given, and subsequently displacements of a myocardial tissue are two-dimensionally measured by tracking processing with pattern matching of the pieces of B mode image data. Then, “strains” and “strain rates” of the myocardial tissue are measured based on a spatial gradient and a time variation of the displacements. Furthermore, the maximum strains and the maximum strain rates are extracted from time series strains and strain rates measured based on B mode image data during an arbitrary period in a diastole. Then, respective representing values of the maximum strain and the maximum strain rate are set in each of plural segments set in the two-dimensional maximum strains and maximum strain rates extracted with regard to the mutually different two motor loads. Then, comparison image data is generated using comparison parameters calculated based on the respective representing values.

### (Configuration of Apparatus)

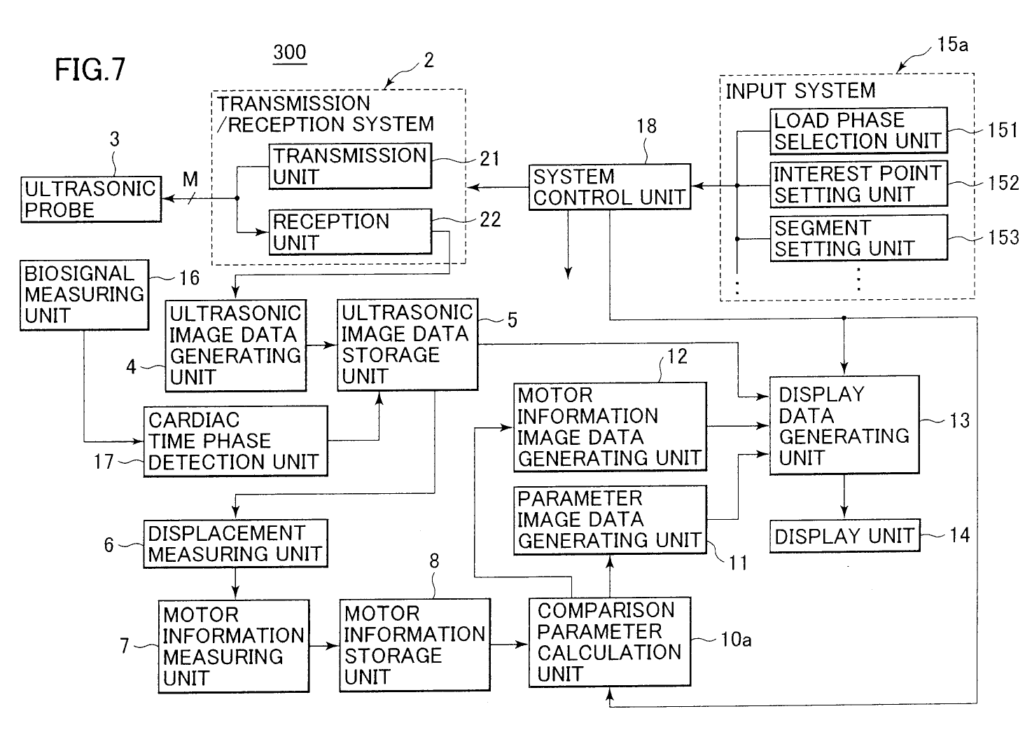

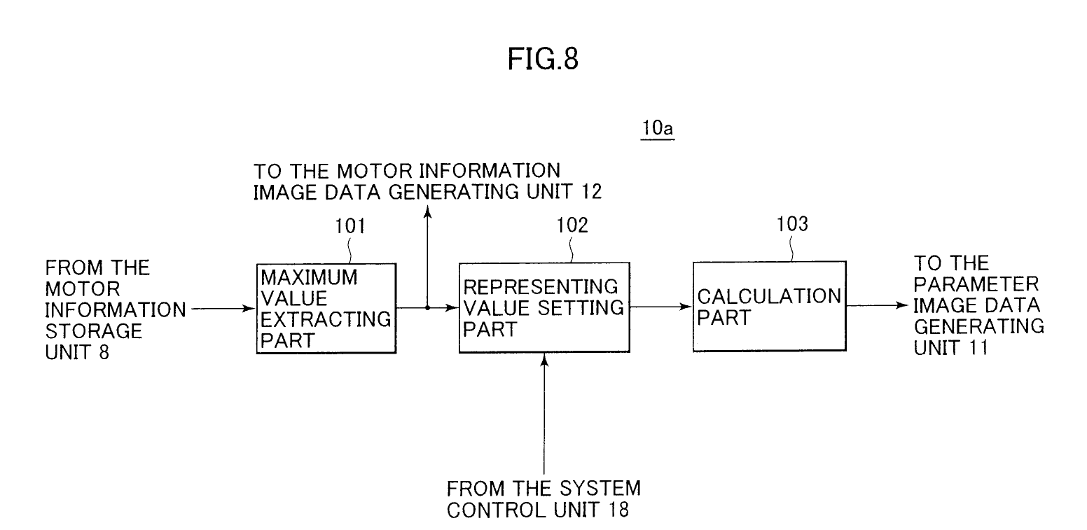

A configuration of an ultrasonic diagnostic apparatus according to the second embodiment of the present invention and fundamental operation of each unit of the apparatus will be described with reference to FIGS. 7 to 10. FIG. 7 is a block diagram showing an overall configuration of the ultrasonic diagnostic apparatus according to the present embodiment. FIG. 8 is a block diagram showing a concrete configuration of a comparison parameter calculation unit included in the ultrasonic diagnostic apparatus. Note that, detail explanation of each unit shown in FIG. 7 having the same configuration and function as that of the ultrasonic diagnostic apparatus 200 in the first embodiment shown in FIG. 1 is omitted with attaching the same sign to the unit.

An ultrasonic diagnostic apparatus 300 according to the present embodiment shown in FIG. 7 includes an ultrasonic probe 3, a transmission/reception system 2, an ultrasonic image data generating unit 4 and an ultrasonic image data storage unit 5. The ultrasonic probe 3 has plural transducers each configured to transmit an ultrasonic pulse (a transmission ultrasonic wave) to an object of which a load phase by motor load is updated sequentially, and convert an ultrasonic reflected wave (a reception ultrasonic wave) acquired from the object into an electric signal (a reception signal). The transmission/reception system 2 is configured to supply drive signals, to the transducers of the ultrasonic probe 3, for transmitting ultrasonic pulses toward a predetermined direction in the object, and to phase and add reception signals respectively corresponding to plural channels, obtained from the transducers. The ultrasonic image data generating unit 4 is configured to perform signal processing of the phased and added reception signals to generate B mode image data. The ultrasonic image data storage unit 5 is configured to add information regarding load phase and cardiac time phase information of a diastole or a systole supplied from the cardiac time phase detection unit 17 to the time-series B mode image data outputted from the ultrasonic image data generating unit 4 and to store the time-series B mode image data.

The ultrasonic diagnostic apparatus 300 also includes a displacement measuring unit 6, a motor information measuring unit 7, a motor information storage unit 8 and a comparison parameter calculation unit 10a. The displacement measuring unit 6 is configured to measure local displacements of myocardial tissue on the respective time-series pieces of B mode data supplied from the ultrasonic image data storage unit 5. The motor information measuring unit 7 is configured to measure “strains” and “strain rates” of the myocardial tissue as motor information based on spatial gradients and temporal variations of the above-mentioned displacements. The motor information storage unit 8 is configured to add the load phase information and the cardiac time phase information to the above-mentioned motor information acquired two-dimensionally with corresponding to the respective time-series pieces of the B mode image data and to store the motor information. The comparison parameter calculation unit 10a is configured to extract “the maximum strain” and “the maximum strain rate” each representing the maximum value or the minimum value from time-series “strains” and “strain rates” at a predetermined portion during an arbitrary period in a diastole in each of the first load phase ni and the second load phase nj read from the motor information storage unit 8, and to calculate comparison parameters based on respective representing values of “the maximum strain” and “the maximum strain rate” in each of plural segments set in the two-dimensional “maximum strains” and “maximum strain rates”.

The ultrasonic diagnostic apparatus 300 also includes a parameter image data generating unit 11, a motor information image data generating unit 12, a display data generating unit 13, a display unit 14, an input system 15a, a bio-signal measuring unit 16, the cardiac time phase detection unit 17 and a system control unit 18. The parameter image data generating unit 11 is configured to generate parameter image data based on a comparison parameter calculated by the above-mentioned comparison parameter calculation unit 10a. The motor information image data generating unit 12 is configured to generate motor information image data using the two-dimensional “maximum strains” and/or “maximum strain rates” extracted by the comparison parameter calculation unit 10a. The display data generating unit 13 is configured to generate display data by overlaying ultrasonic image data with the parameter image data. The display unit 14 is configured to display the display data and/or the motor information image data. The input system 15 is configured to perform inputting of object information, setting of respective conditions for generating ultrasonic image data, parameter image data and display data, selection of a load phase ni and a load phase nj, selection of estimation function of extension ability, setting of segmented regions, inputting of various command signals and the like. The bio-signal measuring unit 16 is configured to measure an ECG wave form of an object. The cardiac time phase detection unit 17 is configured to detect a cardiac time phase during a diastole or a systole using an R wave on an ECG wave form as a reference. The system control unit 18 is configured to overall-control the above-mentioned respective units.

Subsequently, a concrete configuration of the above-mentioned comparison parameter calculation unit 10a will be described with reference to FIG. 8. As shown in FIG. 8, the comparison parameter calculation unit 10a includes a maximum value extracting part 101, a representing value setting part 102 and a calculation part 103.

The maximum value extracting part 101 is configured to extract time-series “strain rates” SR(i, t, x, y) and “strain rates” SR(j, t, x, y) attaching information indicating either the load phase ni or the load phase nj and cardiac time phase information for an arbitrary period during a diastole as incidental information and time-series “strains” S(j, t, x, y) attaching information indicating the load phase nj and cardiac time phase information for the arbitrary period as incidental information, from the motor information stored in the motor information storage unit 8, based on selection information of the load phase ni and the load phase nj (that is, the load phase 1 and the load phase 2 by motor load) and selection information of the estimation function of extension ability supplied from the load phase selection unit 151 included in the input system 15a.

Then, the maximum value extracting part 101 is configured to extract “the maximum strain rates” SRmax(i, x, y), “the maximum strain rates” SRmax(j, x, y) and “the maximum strains” Smax(j, x, y) each representing the maximum value or the minimum value in the time direction from the respective time-series “strain rates” SR(i, t, x, y), “strain rates” SR(j, t, x, y) and “strains” S(j, t, x, y) measured at the interest points Cg(x, y).

The representing value setting part 102 is configured to set plural segmented regions in each of the above-mentioned two-dimensional “maximum strain rates” SRmax(i, x, y), “maximum strain rates” SRmax(j, x, y) and “maximum strains” Smax(j, x, y), based on setting information of segmented regions supplied from the segment setting unit 153 included in the input system 15a, and to set representing values (i.e., a representing value SRmax(i) of the maximum strain rates SRmax(i, x, y), a representing value SRmax(j) of the maximum strain rates SRmax(j, x, y) and a representing value Smax(j) of the maximum strains Smax(j, x, y)) of the plural “maximum strains” and “maximum strain rates” included in the respective segmented regions.

Specifically, respective average values or respective median values (medians) of the two-dimensional “maximum strains” and “maximum strain rates” included in each segmented region are set as the above-mentioned representing values. Then, the calculation part 103 is configured to calculate common comparison parameters K11 to K13 with regard to each segmented region by substituting the above-mentioned representing values set by the representing value setting part 102 in the expression (4) or the expression (5) hereinafter prescribed.

$$\begin{matrix}K 11 = \frac{\underline{S max} ( j ) \cdot \underline{SR max} ( j )}{\underline{SR max} ( i )} \\ K 12 = \underline{S max} ( j ) \cdot \{ \underline{SR max} ( j ) - \underline{SR max} ( i ) \} \\ K 13 = \frac{\underline{S max} ( j ) \cdot \{ \underline{SR max} ( j ) - \underline{SR max} ( i ) \}}{\underline{SR max} ( i )} & ( 4 ) \\ K 11 = \frac{\underline{SR max} ( j )}{\underline{SR max} ( i )} \\ K 12 = \underline{SR max} ( j ) - \underline{SR max} ( i ) \\ K 13 = \frac{\underline{SR max} ( j ) - \underline{SR max} ( i )}{\underline{SR max} ( i )} & ( 5 )\end{matrix}$$

Back to FIG. 8, the motor information image data generating unit 12 is configured to generate motor information image data used for setting plural segmented regions in the above-mentioned “maximum strain rates” SRmax(i, x, y), “maximum strain rates” SRmax(j, x, y) and “maximum strains” Smax(j, x, y), using the two-dimensional “maximum strains” or “maximum strain rates” extracted by the maximum value extracting part 101 included in the comparison parameter calculation unit 10a. Then, the generated motor information image data is displayed on the monitor of the display unit 14 through the display data generating unit 13.

The input system 15a is an interactive interface which includes input devices such as a display panel, a keyboard, various switches, selection buttons, a mouse and a trackball. The input system 15a includes a load phase selection unit 151, an interest point setting unit 152 and a segment setting unit 153. The load phase selection unit 151 is configured to select desired load phase ni and load phase nj from plural load phases. The interest point setting unit 152 is configured to set interest points at predetermined intervals in a myocardial tissue. The segment setting unit 153 is configured to set plural segmented regions and/or regions of interest in the motor information image data generated by the motor information image data generating unit 12 and displayed on the display unit 14. Inputting of object information, setting of respective conditions for generating ultrasonic image data, parameter image data and display data, selection of estimation function of extension ability, setting of a threshold to an ECG waveform, selection of a method for setting representing values, inputting of various command signals and the like are also performed using the above-mentioned input devices and/or display panel included in the input system 15a.

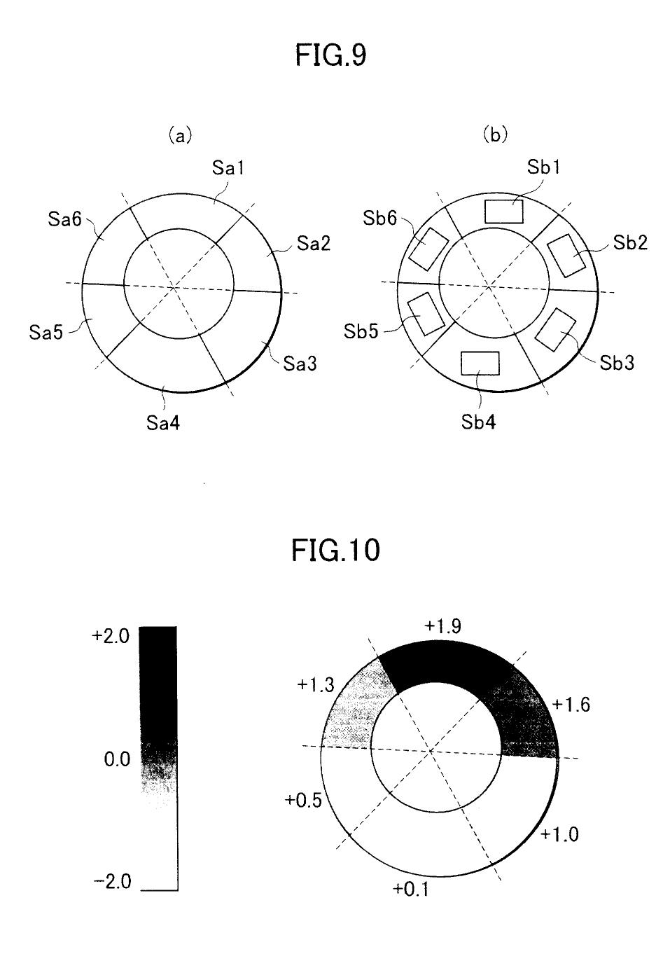

FIG. 9 shows a concrete example of segmented regions and regions of interest set in a myocardial tissue on a cardiac short axis image. FIG. 9(a) shows segmented regions Sa1 to Sa6 set by ASE (American Society of Echocardiography) segmentation used officially in normal ultrasonic diagnostics and FIG. 9(b) shows regions of interest Sb1 to Sb6 set in accordance with the ASE segmentation.

Meanwhile, FIG. 10 shows a concrete example of parameter image data based on representing values of the segmented regions S1 to S6 set by the above-mentioned ASE segmentation. The respective segmented regions S1 to S6 are displayed with colors corresponding to pixel values which the segmented regions have, moreover, displayed with overlaying the pixel values. By performing the display method like this, for example, even the case where a displacement is generated between the B mode image data in the load phase ni and the B mode image data in the load phase nj due to a respiratory movement of an object and the like, stable parameter image data can be generated with less influence from the displacement.

### (Procedure for Displaying Parameter Image Data)

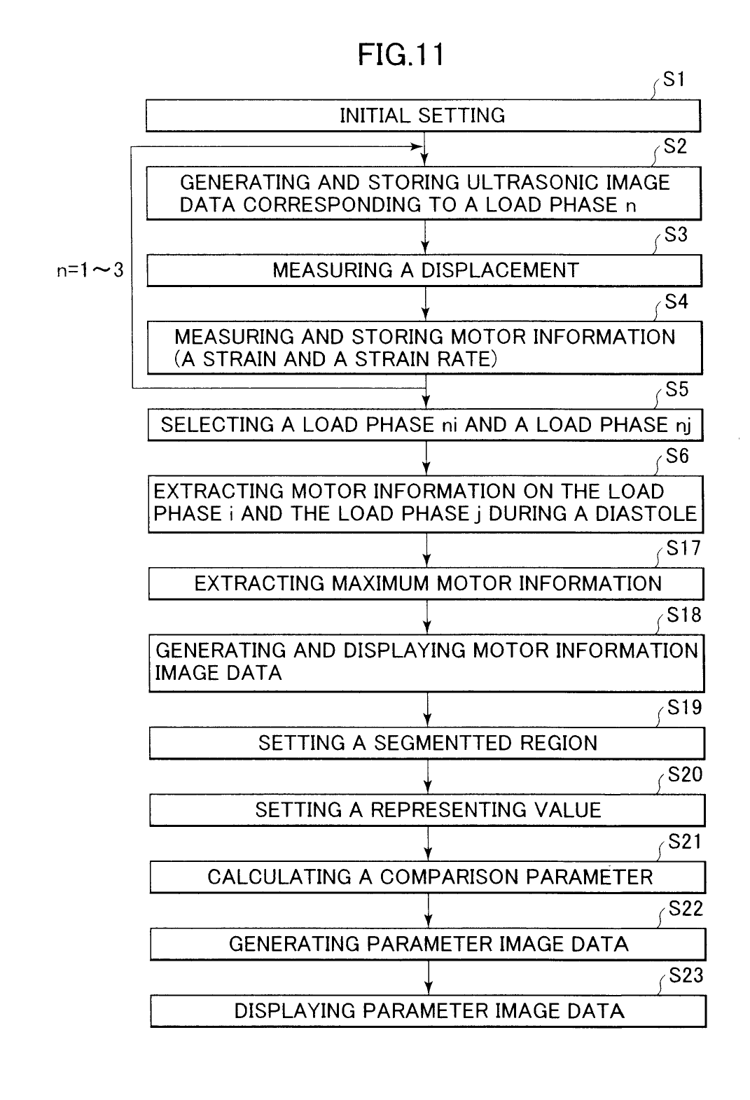

Then, a procedure for displaying parameter image data in the present embodiment will be described with reference to the flowchart on FIG. 11. Note that, explanation of each procedure (step) of FIG. 11 indicating the same procedure as that of the procedure for displaying parameter image data in the first embodiment shown in FIG. 6 is omitted with attaching the same sign to the step.

Specifically, the maximum value extracting part 101 of the comparison parameter calculation unit 10a extracts time-series “strain rates” SR(i, t, x, y) and “strain rates” SR(j, t, x, y) attaching cardiac time phase information during an arbitrary period in a diastole and information indicating either the load phase ni or the load phase nj as incidental information and time-series “strains” S(j, t, x, y) attaching the cardiac time phase information during the arbitrary period and information indicating the load phase nj as incidental information, from the pieces of motor information stored in the motor information storage unit 8, based on selecting information of the load phases and information selecting the estimation function of extension ability supplied through the system control unit 18, similarly to step S6 in FIG. 6 (step S8 in FIG. 11).

Subsequently, the comparison parameter calculation unit 10a extracts “the maximum strain rates” SRmax(i, x, y), “the maximum strain rates” SRmax(j, x, y) and “the maximum strains” Smax(j, x, y), each representing the maximum value or the minimum value in the time direction, from the respective time-series “strain rates” SR(i, t, x, y), “strain rates” SR(j, t, x, y) and “strains” S(j, t, x, y) measured at the plural interest points Cg (x, y) (step S17 in FIG. 11).

On the other hands, the motor information image data generating unit 12 generates motor information image data using the two-dimensional “maximum strains” or “maximum strain rates” extracted by the maximum value extracting part 101 included in the comparison parameter calculation unit 10a and displays the obtained motor information image data on the monitor of the display unit 14 through the display data generating unit 13 (step S18 in FIG. 11).

The operator who observed the motor information image data displayed on the display unit 14 sets plural segmented regions in the motor information image data using the segment setting unit 153 included in the input system 15a (step S19 in FIG. 11). The representing value setting part 102 included in the comparison parameter calculation unit 10a sets plural segmented regions in each of the two-dimensional “maximum strain rates” SRmax(i, x, y), “maximum strain rates” SRmax(j, x, y) and “maximum strains” Smax(j, x, y), based on setting information of the segmented regions supplied from the segment setting unit 153. Then, the representing value setting part 102 sets respective representing values (i.e., a representing value SRmax(i) of “the maximum strain rates” SRmax(i, x, y), a representing value SRmax(j) of “the maximum strain rates” SRmax(j, x, y) and a representing value Smax(j) of “the maximum strains” Smax(j, x, y)) in the plural “maximum strains” and “maximum strain rates” included in each segmented region (step S20 in FIG. 11).

Subsequently, the calculation part 103 included in the comparison parameter calculation unit 10a calculates common comparison parameters K11 to K13 in each segmented region by substituting the above-mentioned representing values set by the representing value setting part 102 in the expression (4) or the expression (5) (step S21 in FIG. 11). The parameter image data generating unit 11 generates parameter image data using either of the above-mentioned comparison parameters K11 to K13 calculated by the comparison parameter calculation unit 10a (step S22 in FIG. 11).

Meanwhile, the display data generating unit 13 generates display data by overlaying B mode image data supplied from the ultrasonic image data storage unit 5 on the comparison parameter image data supplied from the parameter image data generating unit 11 and displays the obtained display data on the monitor of the display unit 14 (step S23 in FIG. 11).

According to the second embodiment mentioned above, a quantitative estimation with regard to extension ability of a myocardial tissue can be performed with high accuracy by generating parameter image data based on myocardial local strain rate information, or strain rate information and strain information obtained in the load phase ni and the load phase nj of the object to which a stress echo method is applied, similarly to the above-mentioned first embodiment

Especially, since a comparison parameter is calculated using “the maximum strain” or, the maximum strain rate” and “the maximum strain” extracted from time-series “strain rates” and “strains” and desired parameter image data is generated based on the comparison parameter in the above-mentioned embodiment, stable parameter image data can be obtained.

Furthermore, by setting representing values of “the maximum strains” and “the maximum strain rates” in each of plural segments set in the two-dimensional “maximum strains” and “maximum strain rates” extracted corresponding to two mutually different motor loads and generating comparison image data using the comparison parameters calculated based on the representing values, even the case where a displacement is generated between the B mode image data in the load phase ni and the B mode image data in the load phase nj due to a respiratory movement of an object and the like, stable parameter image data can be generated with less influence of the displacement.

### 3. Third Embodiment

Subsequently, the third embodiment of the present invention will be described below. An image data generating apparatus in the third embodiment acquires ultrasonic image data generated in advance with regard to an object to which plural mutually different motor loads have been sequentially given through a high-capacity storage media or a network and two-dimensionally measures displacements of a myocardial tissue in pieces of ultrasonic image data corresponding to load phase ni and load phase nj selected from the acquired ultrasonic image data. Then, “strains” and “strain rates” of the myocardial tissue are measured based on a spatial gradient and a time variation of the displacements. Furthermore, the maximum strains and the maximum strain rates are extracted from time series strains and strain rates measured based on B mode image data during an arbitrary period in a diastole. Then, comparison parameters are calculated using the above-mentioned maximum strains and maximum strain rates or respective representing values in plural segments set in the maximum strains and maximum strain rates, and subsequently, comparison image data is generated based on the comparison parameters.

### (Configuration of Apparatus)

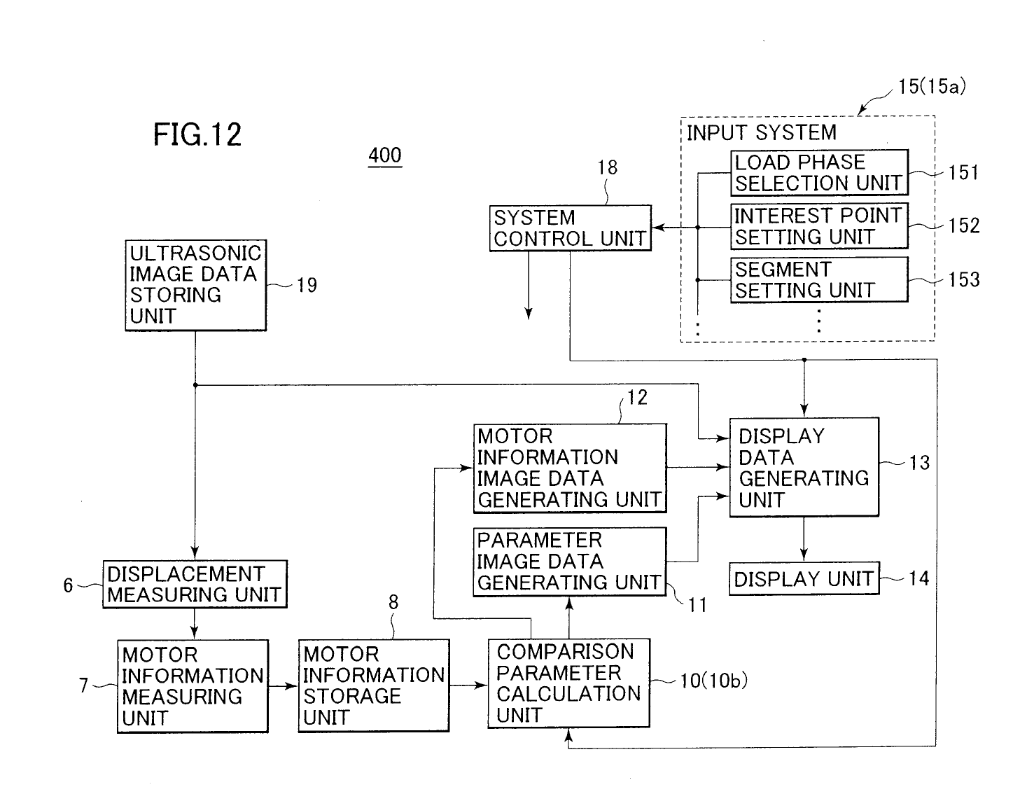

A configuration of the image data generating apparatus according to the third embodiment of the present invention will be described with reference to FIG. 12. FIG. 12 is a block diagram showing an overall configuration of the image data generating apparatus according to the present embodiment. Note that, detail explanation of each unit shown in FIG. 12 having the same configuration and function as that of the ultrasonic diagnostic apparatus 200 in the first embodiment shown in FIG. 1 or the ultrasonic diagnostic apparatus 300 in the second embodiment shown in FIG. 7 is omitted with attaching the same sign to the unit.

Specifically, the image data generating apparatus 400 shown in FIG. 12 includes an ultrasonic image data storing unit 19, a displacement measuring unit 6, a motor information measuring unit 7 and a comparison parameter calculation unit 10 (10a). The ultrasonic image data storing unit 19 stores time series pieces of ultrasonic image data, generated with regard to an object of which a load phase by motor load have been updated sequentially, with load phases and cardiac time phases as incidental information in advance. The displacement measuring unit 6 is configured to measure local displacements of a myocardial tissue in each of time-series pieces of ultrasonic image data, corresponding to a load phase ni and a load phase nj, extracted from the ultrasonic image data storing unit 19. The motor information measuring unit 7 is configured to measure “strains” and “strain rates” of the myocardial tissue as motor information based on spatial gradients and temporal variations of the above-mentioned displacements. The comparison parameter calculation unit 10 (10a) is configured to extract “the maximum strains” and “the maximum strain rates” each representing the maximum value or the minimum value from time-series “strains” and “strain rates” at a predetermined portion during an arbitrary period in a diastole in each of the load phase ni and the load phase nj supplied from the motor information measuring unit 7, and to calculate comparison parameters using the two-dimensional “maximum strains” and “maximum strain rates” or respective representing values of “the maximum strains” and “the maximum strain rates” in each of plural segments set in the “maximum strains” and “maximum strain rates”.

The image data generating apparatus 400 also includes a parameter image data generating unit 11, a motor information image data generating unit 12, a display data generating unit 13, a display unit 14, an input system 15 (15a) and a system control unit 18. The parameter image data generating unit 11 is configured to generate parameter image data based on a comparison parameter calculated by the above-mentioned comparison parameter calculation unit 10 (10a). The motor information image data generating unit 12 is configured to generate motor information image data using the two-dimensional “maximum strains” and/or “maximum strain rates” extracted by the comparison parameter calculation unit 10 (10a). The display data generating unit 13 is configured to generate display data by overlaying ultrasonic image data with the parameter image data. The display unit 14 is configured to display the display data and/or the motor information image data. The input system 15 (15a) is configured to perform inputting of object information, setting of respective conditions for generating parameter image data and display data, selection of a load phase ni and a load phase nj, selection of estimation function of extension ability, setting of segmented regions, inputting of various command signals and the like. The system control unit 18 is configured to overall-control the above-mentioned respective units.

### (Procedure for Displaying Parameter Image Data)

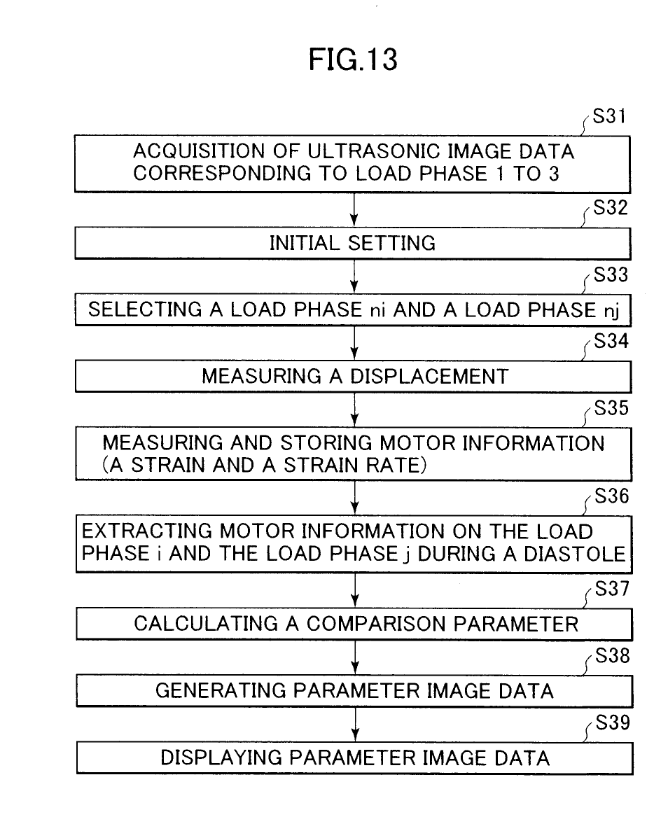

Then, a procedure for displaying parameter image data in the present embodiment will be described with reference to the flowchart on FIG. 13.

Prior to generating parameter image data, time-series pieces of ultrasonic image data, acquired by an ultrasonic diagnostic apparatus not shown in the figure, with regard to an object of which a load phase by motor load is updated sequentially (from the load phase 1 to the load phase 3), is stored with attaching load phases and cardiac time phases in the ultrasonic image data storing unit 19 of the image data generating apparatus 400 preliminarily (step S31 in FIG. 13).

Then, the operator of the image data generating apparatus 400 inputs patient information to the apparatus through the input system 15 (15a), and subsequently, performs initial setting such as setting of respective conditions for generating parameter image data and display data, selection of estimation function of extension ability, setting a threshold value corresponding to “the maximum strain” and the like (step S32 in FIG. 13). Then, the operator selects a load phase ni and a load phase nj with the load phase selection unit 151 included in the input system 15 (15a) (step S33 in FIG. 13).

The displacement measuring unit 6, to which the selecting information is supplied through the system control unit 18, sequentially extracts two pieces of ultrasonic image data adjacent in the time direction from time-series pieces of ultrasonic image data corresponding to the load phase ni and the load phase nj stored in the ultrasonic image data storing unit 19 and measures movement distances (displacements) of a myocardial tissue shown on the pieces of ultrasonic image data by tracking processing for which respective plural interest points Cg set on the pieces of image data are set to centers (step S34 in FIG. 13).

Subsequently, the motor information measuring unit 7 measures local “strains” based on spatial gradients of the displacements measured in the displacement measuring unit 6 and measures “strain rates” based on temporal variations of the “strains” measured with regard to the respective time-series pieces of the B mode image data. Then, the measured time-series “strains” and “strain rates” corresponding to the load phase ni and the load phase nj are stored with cardiac time phases and load phases as incidental information in the motor information storage unit 8 (step S35 in FIG. 13).

When measuring and storing the pieces of motor information corresponding to the load phases ni and nj by motor load are completed, the comparison parameter calculation unit 10 (10a) extracts the time-series “strain rates” corresponding to the load phases ni and nj and the time-series “strains” corresponding to the load phase nj, each attaching cardiac time phase during an arbitrary period in a diastole as incidental information, from the pieces of motor information stored in the motor information storage unit 8, based on information selecting the estimation function of extension ability supplied through the system control unit 18 (step S36 in FIG. 13).

Subsequently, the comparison parameter calculation unit 10 extracts “the maximum strain rates” corresponding to the load phases ni and nj and “the maximum strains” corresponding to the load phase nj, each representing the maximum value or the minimum value in the time direction, from the above-mentioned time-series “strain rates” and “strains”. Then, the comparison parameter calculation unit 10 calculates comparison parameters K1(x, y) to K3(x, y) or K11 to K13 using the two-dimensional “maximum strains” and “maximum strain rates” or respective representing values of the “maximum strains” and “maximum strain rates” in each of plural segments set in the “maximum strains” and “maximum strain rates” (step S37 in FIG. 13). Then, the parameter image data generating unit 11 generates parameter image data using at least one of the above-mentioned comparison parameters calculated two-dimensionally by the comparison parameter calculation unit 10 (10a) (step S38 in FIG. 13).

Meanwhile, the display data generating unit 13 compares each of the two-dimensional “maximum strains” supplied from the comparison parameter calculation unit 10 (10a) to the predetermined threshold supplied from the input system 15 (15a) and performs color transformation of each pixel value (comparison parameter) of the parameter image data corresponding to “the maximum strain” more than the threshold, based on a predetermined pixel value-color transformation format. Additionally, the display data generating unit 13 transforms each pixel value of the parameter image data corresponding to “the maximum strain” not more than the threshold, based on another transformation format. Then, the display data generating unit 13 generates display data by overlaying B mode image data supplied from the ultrasonic image data storage unit 5 on the comparison parameter image data after color transformation and displays the obtained display data on the monitor of the display unit 14 (step S39 in FIG. 13).

According to the third embodiment mentioned above, a quantitative estimation with regard to extension ability of a myocardial tissue can be performed with high accuracy by generating parameter image data based on myocardial local strain rate information, or strain rate information and strain information obtained in the load phase ni and the load phase nj of the object to which a stress echo method is applied

Especially, since a comparison parameter is calculated using the maximum strain rate” and “the maximum strain” extracted from time-series “strain rates” and “strains” and desired parameter image data is generated based on the comparison parameter in the above-mentioned embodiment, stable parameter image data can be obtained.

Further, a normal myocardial tissue and a myocardial tissue with a decreased extension ability can be also observed specifically and easily by performing color display of parameter image data based on a pixel value-color transformation format with a cold color and a warm color corresponding to signs of the comparison parameters.

Additionally, it is possible to distinctly observe a normal myocardial tissue, a myocardial tissue with a decreased extension ability due to a mild or moderate ischemia and the like and a myocardial tissue of which contractile ability is decreased drastically due to a severe ischemia, total necrosis and the like by performing color display of each pixel value (comparison parameter) of parameter image data corresponding to “the maximum strain” more than a predetermined threshold, based on a predetermined pixel value-color transformation format and of each pixel value of the parameter image data corresponding to “the maximum strain” not more than the threshold, based on another transformation format with comparing the above-mentioned “maximum strain” with the threshold.

Furthermore, by setting representing values of “the maximum strains” and “the maximum strain rates” in each of plural segments set in the two-dimensional “maximum strains” and “maximum strain rates” extracted corresponding to two mutually different motor loads and generating comparison image data using the comparison parameters calculated based on the representing values, even the case where a displacement is generated between the B mode image data in the load phase ni and the B mode image data in the load phase nj due to a respiratory movement of an object and the like, stable parameter image data can be generated with less influence of the displacement.

Furthermore, since the image data generating apparatus according to the above-mentioned embodiment can generate parameter image data efficient for a cardiac function examination by using time-series pieces of ultrasonic image data corresponding to plural load phases supplied through a network and the like from an ultrasonic diagnostic apparatus set separately, an operator can perform diagnostic of the object efficiently with less time and place constraints.

Hereinbefore, embodiments of the present invention have been described, the present invention is not limited to the above-mentioned embodiment and can be performed with modification. For example, in the above-mentioned embodiment, though the case of acquiring B mode image data as ultrasonic image data from an object to which a motor load is given and generating desired parameter image data based on the B mode image data has been described, a drug load may be used instead of a motor load and tissue Doppler image data and/or color Doppler image data may be acquired as ultrasonic image data.

In addition, though the case where the cardiac time phase detection unit 17 detects cardiac time phases during a diastole or a systole based on an ECG waveform of the object measured by the biosignal measuring unit 16 has been described in the above-mentioned embodiment, the above-mentioned cardiac time phases may be detected by measuring an area change in a cardiac chamber displayed on ultrasonic image data such as B mode image data.

In addition, the case of measuring displacements of a myocardial tissue by performing tracking processing of pieces of B mode image data adjacent in the time direction regarding the displacement measuring unit 6 has been described. However not be limited to, for example, detecting displacements of a myocardial tissue by temporal integration of rate information shown on the above-mentioned tissue Doppler image data is also allowed.

Further, though the case of generating parameter image data based on ultrasonic image data generated in a load phase 1 and a load phase 2 by motor load has been described, generating parameter image data based on ultrasonic image data acquired in another load phase may be performed.

What is claimed is:

1. An ultrasonic diagnostic apparatus which generates parameter image data useful for functional diagnosis of a myocardial tissue based on time series pieces of ultrasonic image data obtained by transmitting and receiving ultrasonic waves to and from an object to which plural mutually different motor or drug loads are sequentially given, the apparatus comprising:
a displacement measuring unit configured to two-dimensionally measure displacements of a myocardial tissue in pieces of ultrasonic image data adjacent in time direction;
a motor information measuring unit configured to measure strains and strain rates of the myocardial tissue based on the displacements as motor information;
a comparison parameter calculation unit configured to calculate comparison parameters based on the strain rates, or the strains and strain rates; and
a parameter image data generating unit configured to generate parameter image data using the comparison parameters.

2. An ultrasonic diagnostic apparatus of claim 1,
wherein said displacement measuring unit is configured to measure the displacements of the myocardial tissue by performing pattern matching of the pieces of the ultrasonic image data adjacent in the time direction

3. An ultrasonic diagnostic apparatus of claim 1,
wherein said motor information measuring unit is configured to measure the strains based on spatial gradients of the two-dimensional time-series displacements measured by said displacement measuring unit and measure the strain rates based on time variations in the strains.

4. An ultrasonic diagnostic apparatus of claim 1,
wherein said comparison parameter calculation unit includes:
a maximum value extracting unit configured to extract maximum strain rates from the time-series strain rates measured by said motor information measuring unit during an arbitrary period in diastole of a heart; and
a calculation unit configured to calculate the comparison parameters based on the maximum strain rates under the mutually different motor or drug loads.

5. An ultrasonic diagnostic apparatus of claim 1,
wherein said comparison parameter calculation unit includes:
a maximum value extracting unit configured to respectively extract maximum strain rates and maximum strains from the time-series strain rates and strains measured by said motor information measuring unit during an arbitrary period in diastole of a heart; and
a calculation unit configured to calculate the comparison parameters based on the maximum strain rates and the maximum strains under the mutually different motor or drug loads.

6. An ultrasonic diagnostic apparatus of claim 1,
wherein said comparison parameter calculation unit includes:
a maximum value extracting unit configured to extract maximum strain rates from the time-series strain rates measured by said motor information measuring unit during an arbitrary period in diastole of a heart;
a representing value setting unit configured to set representing values of the maximum strain rates in plural respective segmented regions set to the two-dimensional maximum strain rates extracted by said maximum value extracting unit; and
a calculation unit configured to calculate the comparison parameters based on the representing values of the maximum strain rates under the mutually different motor or drug loads.

7. An ultrasonic diagnostic apparatus of claim 1,
wherein said comparison parameter calculation unit includes:
a maximum value extracting unit configured to respectively extract maximum strain rates and maximum strains from the time-series strain rates and strains measured by said motor information measuring unit during an arbitrary period in diastole of a heart;
a representing value setting unit configured to set representing values of each of the maximum strain rates and the maximum strains in plural respective segmented regions set to the two-dimensional maximum strain rates and maximum strains respectively extracted by said maximum value extracting unit; and
a calculation unit configured to calculate the comparison parameters based on the representing values of each of the maximum strain rates and the maximum strains under the mutually different motor or drug loads.

8. An ultrasonic diagnostic apparatus of claim 5,
wherein said parameter image data generating unit is configured to generate display data by comparing the maximum strains extracted by said maximum value extracting unit with a predetermined threshold and performing color-conversion of pixels corresponding to maximum strains larger than the predetermined threshold and pixels corresponding to maximum strains smaller than the predetermined threshold according to mutually different conversion formats.

9. An ultrasonic diagnostic apparatus of claim 8,
wherein said parameter image data generating unit is configured to generate the display data by superposing the parameter image data after the color-conversion on a piece of the ultrasonic image data.

10. An ultrasonic diagnostic apparatus of claim 6, further comprising:
a motor information image data generating unit configured to generate motor information image data based on the maximum strain rates extracted by said maximum value extracting unit; and
a segment setting unit configured to set the segmented regions in the two-dimensional maximum strain rates using the motor information image data.

11. An ultrasonic diagnostic apparatus of claim 7,
a motor information image data generating unit configured to generate motor information image data based on at least one of the maximum strain rates and the maximum strains extracted by said maximum value extracting unit; and
a segment setting unit configured to set the segmented regions in the two-dimensional maximum strain rates and maximum strains respectively using the motor information image data.

12. An ultrasonic diagnostic apparatus of claim 4,
wherein said comparison parameter calculation unit is configured to calculate the comparison parameters using at least one of subtraction values and division values between the maximum strain rates under the mutually different motor or drug loads.

13. An image data generating apparatus which generates parameter image data useful for functional diagnosis of a myocardial tissue based on time series pieces of ultrasonic image data obtained by transmitting and receiving ultrasonic waves to and from an object to which plural mutually different motor or drug loads are sequentially given, the apparatus comprising:
an ultrasonic image data storing unit configured to store the time series pieces of the ultrasonic image data together with information regarding cardiac time phase and information regarding load state;
a displacement measuring unit configured to two-dimensionally measure displacements of a myocardial tissue in pieces of ultrasonic image data adjacent in time direction, read from said ultrasonic image data storing unit;
a motor information measuring unit configured to measure strains and strain rates of the myocardial tissue based on the displacements as motor information;
a comparison parameter calculation unit configured to calculate comparison parameters based on the strain rates, or the strains and strain rates; and
a parameter image data generating unit configured to generate parameter image data using the comparison parameters.

14. An ultrasonic diagnostic method for generating parameter image data useful for functional diagnosis of a myocardial tissue based on time series pieces of ultrasonic image data obtained by transmitting and receiving ultrasonic waves to and from an object to which plural mutually different motor or drug loads are sequentially given, the method comprising:
two-dimensionally measuring displacements of a myocardial tissue in pieces of ultrasonic image data adjacent in time direction;
measuring strains and strain rates of the myocardial tissue based on the displacements as motor information;
calculating comparison parameters based on the strain rates, or the strains and strain rates; and
generating parameter image data using the comparison parameters.

15. An ultrasonic diagnostic method of claim 14,
wherein the displacements of the myocardial tissue are measured by pattern matching of the pieces of the ultrasonic image data adjacent in the time direction.

16. An ultrasonic diagnostic method of claim 14,
wherein the strains are measured based on spatial gradients of the two-dimensional time-series displacements and the strain rates are measured based on time variations in the strains.

17. An ultrasonic diagnostic method of claim 14,
wherein said calculating the comparison parameters includes:
extracting maximum strain rates from the time-series strain rates during an arbitrary period in diastole of a heart; and
obtaining the comparison parameters based on the maximum strain rates under the mutually different motor or drug loads.

18. An ultrasonic diagnostic method of claim 14,
wherein said calculating the comparison parameters includes:
respectively extracting maximum strain rates and maximum strains from the time-series strain rates and strains during an arbitrary period in diastole of a heart; and
obtaining the comparison parameters based on the maximum strain rates and the maximum strains under the mutually different motor or drug loads.

19. An ultrasonic diagnostic method of claim 14,
wherein said calculating the comparison parameters includes:
extracting maximum strain rates from the time-series strain rates during an arbitrary period in diastole of a heart;
setting representing values of the maximum strain rates in plural respective segmented regions set to the two-dimensional maximum strain rates; and
obtaining the comparison parameters based on the representing values of the maximum strain rates under the mutually different motor or drug loads.

20. An ultrasonic diagnostic method of claim 14,
wherein said calculating the comparison parameters includes:
respectively extracting maximum strain rates and maximum strains from the time-series strain rates and strains during an arbitrary period in diastole of a heart;
setting representing values of each of the maximum strain rates and the maximum strains in plural respective segmented regions set to the two-dimensional maximum strain rates and maximum strains; and
obtaining the comparison parameters based on the representing values of each of the maximum strain rates and the maximum strains under the mutually different motor or drug loads.

21. An ultrasonic diagnostic method of claim 18,
wherein display data is generated by comparing the maximum strains with a predetermined threshold and performing color-conversion of pixels corresponding to maximum strains larger than the predetermined threshold and pixels corresponding to maximum strains smaller than the predetermined threshold according to mutually different conversion formats.

22. An ultrasonic diagnostic method of claim 21,
wherein the display data is generated by superposing the parameter image data after the color-conversion on a piece of the ultrasonic image data.

23. An ultrasonic diagnostic method of claim 19,
wherein motor information image data is generated based on the maximum strain rates and the segmented regions are set in the two-dimensional maximum strain rates using the motor information image data.

24. An ultrasonic diagnostic method of claim 20,
wherein motor information image data is generated based on at least one of the maximum strain rates and the maximum strains and the segmented regions are set in the two-dimensional maximum strain rates and maximum strains respectively using the motor information image data.

25. An image data generating method for generating parameter image data useful for functional diagnosis of a myocardial tissue based on time series pieces of ultrasonic image data obtained by transmitting and receiving ultrasonic waves to and from an object to which plural mutually different motor or drug loads are sequentially given, the method comprising:
storing the time series pieces of the ultrasonic image data together with information regarding cardiac time phase and information regarding load state;
two-dimensionally measuring displacements of a myocardial tissue in pieces of ultrasonic image data adjacent in time direction, read from the stored ultrasonic image data;
measuring strains and strain rates of the myocardial tissue based on the displacements as motor information;
calculating comparison parameters based on the strain rates, or the strains and strain rates; and
generating parameter image data using the comparison parameters.
